(19) United States
(12) Patent Application Publication
(10) Pub. No.: US 2025/0269369 A1
(43) Pub. Date: Aug. 28, 2025

(54) COLORIMETRIC SENSOR INCLUDING METAL-ORGANIC FRAMEWORK AND METHOD OF MANUFACTURING THE COLORIMETRIC SENSOR

(71) Applicant: ELECTRONICS AND TELECOMMUNICATIONS RESEARCH INSTITUTE, Daejeon (KR)

(72) Inventors: Dae-Sik LEE, Daejeon (KR); Junyeong LEE, Daejeon (KR); Jungseok HEO, Daejeon (KR); JinSeong OH, Daejeon (KR); Dongmin KIM, Daejeon (KR); SeungYun OH, Daejeon (KR)

(21) Appl. No.: 19/059,009

(22) Filed: Feb. 20, 2025

(30) Foreign Application Priority Data
Feb. 27, 2024 (KR) 10-2024-0028046

(51) Int. Cl. B01L 3/00; G01N 21/78; G01N 33/00

(52) U.S. Cl. CPC B01L 3/502707; G01N 21/78; G01N 33/0027; B01L 2200/12; B01L 2300/0663

(57) ABSTRACT

A method of manufacturing a colorimetric sensor is provided. The method includes a step of obtaining metalloporphyrin, a step of synthesizing the metalloporphyrin into a metal-organic framework, a step of adsorbing the metal-organic framework onto a dye catcher sheet, and a step of drying the dye catcher sheet. According to the method, based on a good color change reactivity of metalloporphyrin and a structural feature (a wide surface area based on an internal empty space) of a metal-organic framework synthesized from the metalloporphyrin, a colorimetric sensor including the metal-organic framework may provide a good color change reactivity on a gas of a low concentration.

### CROSS-REFERENCE TO RELATED APPLICATION

This application claims the benefit of Korean Patent Application No. 10-2024-0028046 filed on Feb. 27, 2024, which is hereby incorporated by reference as if fully set forth herein.

### BACKGROUND

### Field of the Invention

The present disclosure relates to a method of manufacturing a colorimetric sensor, and more particularly, to a method of manufacturing a colorimetric sensor which may detect a gas corresponding to a volatile organic compound.

### Discussion of the Related Art

Colorimetric sensors may be sensors where a color is changed based on a change in a condition or a peripheral environment and may sense the presence of a specific material, a change in temperature, a change in humidity, and a change in pH value among environments, based on a change in color. Colorimetric sensors are referred to as color-change sensors.

Colorimetric sensors may be used in various application fields. For example, colorimetric sensors may be used for evaluating the freshness of food in food industry, and in medical field, colorimetric sensors may be used to monitor a health status of a patient. Also, colorimetric sensors may be used in environment monitoring, industry control, and safety system.

Colorimetric sensors have various operation principles, and the operation principles of colorimetric sensors differ based on a specific material sensed by a colorimetric sensor. Generally, colorimetric sensors react with a specific chemical material to operate based on a chemical reaction which causes a change in color.

Furthermore, pH indicators or solvent-responsive dyes used in conventional colorimetric sensors provide a good color change reactivity in a gas sensing process of a high concentration (for example, 100 ppm (part per million) or more), but do not provide a significant color change in a gas sensing process of a low concentration and have a drawback where quantitative analysis is difficult due to a color change reactivity on peripheral water.

### SUMMARY

An aspect of the present disclosure is directed to providing a method of manufacturing a colorimetric sensor which includes a metal-organic framework synthesized from metalloporphyrin so as to provide a high color change reactivity in a gas of a low concentration and provide a low color change reactivity on peripheral water.

To achieve these and other advantages and in accordance with the purpose of the invention, as embodied and broadly described herein, there is provided a method of manufacturing a colorimetric sensor, the method including: a step of obtaining metalloporphyrin; a step of synthesizing the metalloporphyrin into a metal-organic framework; a step of adsorbing the metal-organic framework onto a dye catcher sheet; and a step of drying the dye catcher sheet.

In an embodiment, the metalloporphyrin may be a compound where porphyrin is bonded to a metal ion, and the metal ion may be one of a Sn(II) ion, a Cu(II) ion, an Fe(II) ion, a Zn(II) ion, a Ni(II) ion, a Mg(II) ion, a Co(II) ion, an Al(III) ion, an Eu(II) ion, and a Pd(II) ion.

In an embodiment, the porphyrin may have a structure where four pyridines are connected to one another in a ring shape, and each of the four pyridines may be a compound including nitrogen (N).

In an embodiment, the step of obtaining the metalloporphyrin may include: a step of preparing a porphyrin as a precursor material; and a step of mixing the porphyrin with a metal compound to obtain the metalloporphyrin, and the metal compound may include one metal of tin (Sn), copper (Cu), iron (Fe), zinc (Zn), nickel (Ni), magnesium (Mg), cobalt (Co), aluminum (Al), europium (Eu), and palladium (Pd).

In an embodiment, the step of synthesizing the metalloporphyrin into the metal-organic framework may include: a step of generating a compound where the metalloporphyrin is mixed with a linker material; a step of adding a catalyst to the compound; and a step of heating the compound with the catalyst added thereto to generate the metal-organic framework.

In an embodiment, the linker material may be zirconium chloride (ZrCl₄).

In an embodiment, the catalyst may be a benzoic acid.

In an embodiment, the step of generating the compound where the metalloporphyrin is mixed with the linker material may include a step of adding zirconium chloride (ZrCl₄), which is the linker material, and a solvent to a vial with the metalloporphyrin put therein and uniformly mixing the zirconium chloride (ZrCl₄), the solvent, and the metalloporphyrin with one another through sonication.

In an embodiment, the solvent may be one of dimethylformamide, methanol, ethanol, acetone, and isopropanol.

In an embodiment, the step of adding the catalyst to the compound may include a step of adding a benzoic acid, which is the catalyst, to a vial where a compound of the metalloporphyrin and zirconium chloride (ZrCl₄) which is the linker material is put, and then, mixing the benzoic acid and the compound with each other through sonication.

In an embodiment, the step of heating the compound with the catalyst added thereto to generate the metal-organic framework may include: a step of sealing, by using a tape, a vial where a compound of the metalloporphyrin, zirconium chloride (ZrCl₄) which is the linker material, and the benzoic acid which is the catalyst is put, and then, performing a reaction therebetween in an oven; and a step of drying a product, obtained through a vacuum filtration apparatus after the vial is cooled up to a room temperature, in an oven to generate the dried product as the metal-organic framework.

In an embodiment, the step of adsorbing the metal-organic framework onto the dye catcher sheet may include: a step of putting the metal-organic framework into a vial and adding a solvent to the vial to disperse the metal-organic framework in the solvent; and a step of putting the dye catcher sheet into the vial where the solvent with the metal-organic framework dispersed therein is put, and then, adsorbing the metal-organic framework onto the dye catcher sheet through magnetic stirring.

In an embodiment, the solvent may be one selected from among methanol, dimethylformamide (DMF) acetone, isopropanol, and ethanol.

In an embodiment, the dye catcher sheet may be a viscose fiber.

In an embodiment, the metal-organic framework may be dispersed in the solvent at a weight ratio of 0.1 wt % to 400 wt % of a weight of the dye catcher sheet.

According to embodiments of the present invention, based on a good color change reactivity of metalloporphyrin and a structural feature (a wide surface area based on an internal empty space) of a metal-organic framework synthesized from the metalloporphyrin, a colorimetric sensor including the metal-organic framework may provide a good color change reactivity on a gas (for example, a gas reacting on a volatile organic compound) of a low concentration (for example, 100 ppb (part per billion)).

Moreover, because the colorimetric sensor including the metal-organic framework is low in color change reactivity on water, a color change sensing error occurring in a peripheral water environment may be reduced.

### BRIEF DESCRIPTION OF THE DRAWINGS

The accompanying drawings, which are included to provide a further understanding of the disclosure and are incorporated in and constitute a part of this application, illustrate embodiments of the disclosure and together with the description serve to explain the principle of the disclosure.

FIG. 1 is a diagram for describing a structural formula representing a molecular structure of metalloporphyrin capable of being used in a colorimetric sensor according to an embodiment of the present invention.

FIG. 2 is a flowchart for describing a method of manufacturing a colorimetric sensor including a metal-organic framework, according to an embodiment of the present invention.

FIG. 3 is a detailed flowchart of step S112 of FIG. 2.

FIG. 4 is a detailed flowchart of step S120 of FIG. 2.

FIG. 5 is a conceptual diagram for describing a process of synthesizing the metalloporphyrin into a metal-organic framework.

FIG. 6 is a photograph image of a metal-organic framework photographed by a scanning microscope, according to an embodiment of the present invention.

FIG. 7 is a photograph image of an enlarged portion A of FIG. 6.

FIG. 8 is a photograph image of a metal-organic framework adsorbed onto a viscose fiber and photographed by a scanning microscope, according to an embodiment of the present invention.

FIG. 9 is a graph showing an RGB color change reactivity with respect to humidity in a general colorimetric sensor manufactured with a color-responsive dye and a colorimetric sensor including a metal-organic framework synthesized from metalloporphyrin according to an embodiment of the present invention.

FIG. 10 is a diagram for describing a colorimetric sensor array according to an embodiment of the present invention.

### DETAILED DESCRIPTION OF THE INVENTION

In the following description, the technical terms are used only for explaining a specific exemplary embodiment while not limiting the present invention. The terms of a singular form may include plural forms unless referred to the contrary. The meaning of ‘comprise’, ‘include’, or ‘have’ specifies a property, a region, a fixed number, a step, a process, an element and/or a component but does not exclude other properties, regions, fixed numbers, steps, processes, elements and/or components.

Hereinafter, embodiments of the present invention will be described in detail with reference to the accompanying drawings.

To manufacture a colorimetric sensor according to an embodiment of the present invention, a material such as metalloporphyrin may be used. Because the metalloporphyrin has properties where a color is changed based on a specific environment condition or a chemical material, the metalloporphyrin may be usefully used as a colorimetric sensor for detecting a gas of a volatile organic compound.

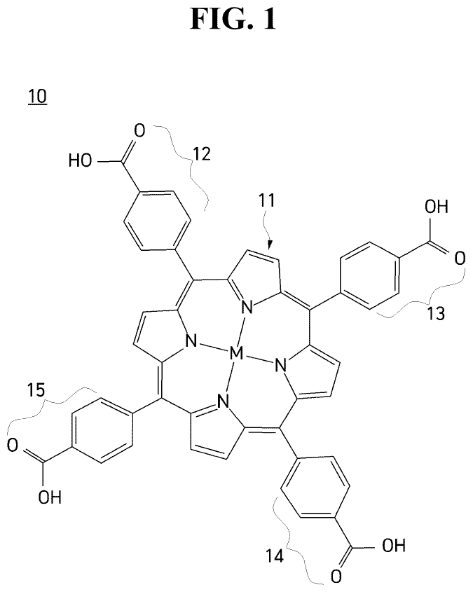

FIG. 1 is a diagram for describing a structural formula representing a molecular structure of a metalloporphyrin 10 used in a colorimetric sensor according to an embodiment of the present invention.

Referring to FIG. 1, the metalloporphyrin 10 according to an embodiment of the present invention may be a compound where a porphyrin 11 is bonded to a metal ion M. Here, the metal ion M may be, for example, one selected from among a Sn(II) ion, a Cu(II) ion, an Fe(II) ion, a Zn(II) ion, a Ni(II) ion, a Mg(II) ion, a Co(II) ion, an Al(III) ion, an Eu(II) ion, and a Pd(II) ion.

The porphyrin 11 may be an organic compound having a structure (hereinafter referred to as a pyridine ring) where four pyridines are connected to one another in a ring shape. Each pyridine may be a compound including nitrogen (N), and a molecular formula thereof may be represented by, for example, CHN.

Four 4-carboxyphenyls 12, 13, 14, and 15 may be bonded to a pyridine ring. As the four 4-carboxyphenyls 12, 13, 14, and 15 are bonded to the pyridine ring, the porphyrin 11 may be represented by meso-tetra(4-carboxyphenyl)porphine (TCCP), and a porphyrin bonded to the metal ion M may be represented by metallo-TCCP.

The metalloporphyrin 10 according to an embodiment of the present invention may be used as a colorimetric sensor which senses a gas corresponding to a volatile organic compound and may provide a significant color change reactivity on a high-concentration gas of 100 ppm or more like pH indicators or solvent-responsive dyes described above, but may not provide a significant color change reactivity on a low-concentration gas, whereby it may be difficult to smoothly sense a gas in a low concentration.

Moreover, because the metalloporphyrin provides a color change reactivity on peripheral water, there may be a drawback where a gas sensing error occurs in a water environment.

Moreover, in a case where a colorimetric sensor is manufactured with the metalloporphyrin, a low-concentration gas of 100 ppb or less may be sensed, but an environmental limitation such as a vacuum state and a high temperature (300° C. or more) may be needed. This may denote that expensive analysis equipment and the cost are needed, and moreover, may not be suitable for biomarker analysis and may largely reduce convenience and mobility which are advantages of a colorimetric sensor.

To solve such a drawback, an embodiment of the present invention may be characterized in that a colorimetric sensor is manufactured with a metal organic framework (MOF) synthesized from the metalloporphyrin so as to provide a significant color change reactivity on a low-concentration gas at a temperature of 100° C. or less.

The MOF may provide various merits. The MOF may have unique porosity and a large surface area. Based on such a structural feature, gas molecules may be effectively adsorbed onto the MOF, and particularly, may be useful for sensing of a colorless gas.

Moreover, the MOF may be configured by a combination of a metal ion and an organic ligand, and thus, the selectivity of a specific gas may increase. Accordingly, it may be effective to distinguish and sense a specific compound or gas.

Moreover, the MOF may be favorable to lighten a colorimetric sensor and may be manufactured in various shapes, and thus, may be good in portability. Such a characteristic may enable a sensor to be easily used in a portable device or various environments.

Moreover, the MOF may be high in stability, and thus, may be used in various environments. This may enable a sensor to effectively operate under various environment conditions. A colorimetric sensor manufactured with the MOF may be used in various application fields. The MOF may be extensively applied in chemical, medical, environmental, energy fields, and particularly, may be used to sense various gases.

Moreover, the MOF may be variously designed through a selection of various metal ions and organic ligands. The MOF may precisely adjust a characteristic of a sensor to obtain a desired characteristic.

Hereinafter, a method of manufacturing a colorimetric sensor including an MOF according to an embodiment of the present invention will be described in detail.

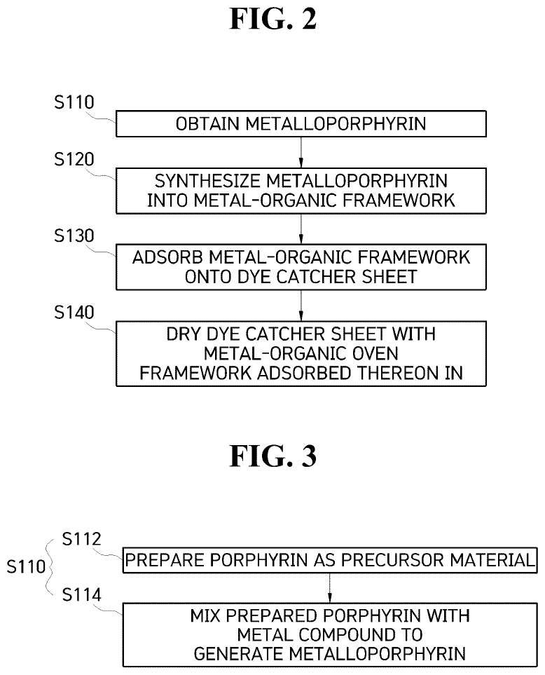

FIG. 2 is a flowchart for describing a method of manufacturing a colorimetric sensor including a metal-organic framework, according to an embodiment of the present invention.

Referring to FIG. 2, first, in step S110, a process of obtaining a metalloporphyrin may be performed.

Subsequently, in step S120, a process of synthesizing the metalloporphyrin into an MOF may be performed.

Subsequently, in step S130, a process of adsorbing the MOF, synthesized from the metalloporphyrin, onto a dye catcher sheet may be performed.

Subsequently, in step S140, a process of drying the dye catcher sheet with the MOF adsorbed thereon may be performed. The dried dye catcher sheet may be a colorimetric sensor.

Hereinafter, each step will be described in more detail.

### Obtain Metalloporphyrin (S110)

FIG. 3 is a detailed flowchart of step S112 of FIG. 2.

Referring to FIG. 3, first, in step S112, a process of preparing a porphyrin as a precursor material (or a start material) may be performed. In a structural formula of FIG. 1, when the metal ion M inserted into a middle is omitted, a structural formula of the porphyrin may be obtained. Here, the porphyrin may be represented by meso-tetra(4-carboxyphenyl)porphine (TCCP).

Subsequently, in step S114, a process of mixing the prepared porphyrin with a metal compound to obtain the metalloporphyrin 11 may be performed. Here, the metal compound may use, for example, SnCl₂, CuCl₂, FeCl₂, ZnCl₂, and Ni(NO₃)₂. However, the present invention is not limited thereto, and a metal compound including magnesium (Mg), cobalt (Co), aluminum (Al), europium (Eu), and palladium (Pd).

### Embodiment

In a case where iron chloride (FeCl₂) of 1.25 mmol to 2.25 mmol (millimole) is used as a metal compound, TCCP of 230 mg to 330 mg (preferably, TCCP of 280 mg), FeCl₂ of 1.25 mmol to 2.25 mmol (millimole) (preferably, FeCl₂ of 1.75 mmol), and a dimethylformamide (DMF) solvent of 20 mL to 30 mL (preferably, 25 mL) may be put into a 70 mL vial and may be uniformly mixed through sonication for 5 minutes.

Subsequently, a process of sealing the vial with a tape and putting the sealed vial into an oven to heat at 100° C. to 140° C. (preferably, 120° C.) for 24 hours, and then, cooling the heated vial up to a room temperature may be performed. Accordingly, a metalloporphyrin 10 may be generated in the vial.

Synthesize Metalloporphyrin into MOF (S120)

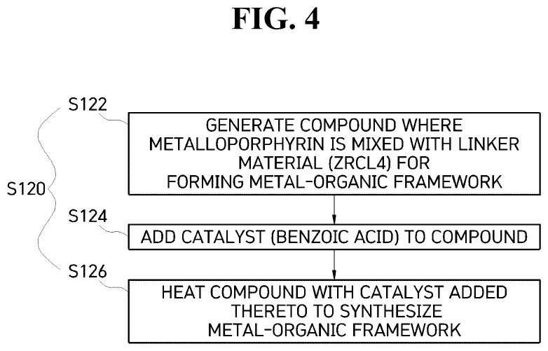

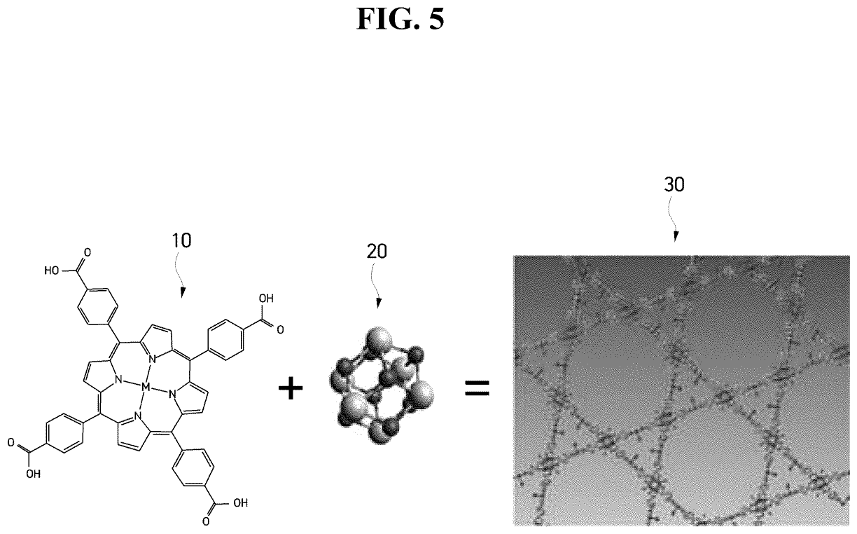

FIG. 4 is a detailed flowchart of step S120 of FIG. 2. FIG. 5 is a conceptual diagram for describing a process of synthesizing a metalloporphyrin into an MOF.

Referring to FIGS. 4 and 5, first, in step S122, a process of mixing the metalloporphyrin 10 with a linker material 20 for forming an MOF to generate a compound may be performed. Here, for example, zirconium chloride (ZrCl₄) may be used as the linker material 20.

In the embodiment of step S122, zirconium chloride (ZrCl₄) of 330 mg to 430 mg (preferably, 380 mg) which is the linker material 20 and a DMF solvent of 10 mL to 20 mL (preferably, 15 mL) may be added to the vial with the metalloporphyrin 10 put therein may be uniformly mixed through sonication for several minutes (for example, 5 minutes). In this case, instead of the DMF solvent, methanol, ethanol, acetone, and isopropanol may be used as a solvent.

Subsequently, in step S124, a process of adding a catalyst to the compound may be performed. Here, the catalyst may be, for example, a benzoic acid.

In the embodiment of step S124, a benzoic acid of 13 g to 14 g (preferably, 13.5 g) may be added to the vial where a compound of the metalloporphyrin 10 and ZrCl₄ is put and may be mixed through sonication for several minutes (for example, 30 minutes).

Subsequently, in step S126, a process of heating the compound with the catalyst added thereto to synthesize an MOF 30 may be performed.

In the embodiment of step S126, a vial where a compound of the metalloporphyrin 10, ZrCl₄ which is the linker material, and the benzoic acid is put may be sealed by a Teflon tape, and then, a reaction may be performed in an oven for 48 hours at 110° C. to 130° C. (preferably, 120° C.). Subsequently, after the vial is cooled up to a room temperature, a product obtained through a vacuum filtration apparatus and methanol may be dried in an oven at 60° C., and thus, an MOF synthesized from the metalloporphyrin 10 may be obtained.

Process of Adsorbing MOF onto Dye Catcher Sheet (S130)

The MOF obtained in step S120 or step S126 described above may be put into a vial, and then, an ethanol solvent may be added to the vial, and the MOF may be dispersed in the ethanol solvent. In this case, instead of the ethanol solvent, methanol, acetone, and isopropanol may be used as a solvent.

Subsequently, the dye catcher sheet may be cut in a specific shape (for example, a circular shape) by a puncher, and the cut dye catcher sheet may be put into the vial where a solvent with ethanol and the MOF dispersed therein is put and may be magnetic-stirred for 24 hours, and thus, the MOF may be adsorbed onto the dye catcher sheet. Here, the dye catcher sheet may be a viscose fiber.

In an embodiment, an MOF of 15 mg to 25 mg (preferably, 20 mg) may be put into a vial of 20 mL, an ethanol solvent of 10 mL to 20 mL (preferably, 15 mL) may be added to the vial, and the MOF may be dispersed in the ethanol solvent through magnetic stirring performed for 12 hours. Subsequently, the viscose fiber may be cut by a puncher, and the cut viscose fiber may be put into the vial and may be magnetic-stirred for 24 hours. In an embodiment, a weight of MOF may be 0.1 wt % to 400 wt % of a weight of the viscose fiber used as a supporter.

Dry Dye Catcher Sheet with MOF Adsorbed Thereon (S140)

The dye catcher sheet with the MOF adsorbed thereon may be unloaded from the vial, and then, when the dye catcher sheet is dried in an oven at 60° C., a colorimetric sensor may be finished. When such a colorimetric sensor is exposed to a specific gas, the specific gas may cause a color change, based on a chemical reaction and a change in polarity occurring when entering an empty space of the MOF.

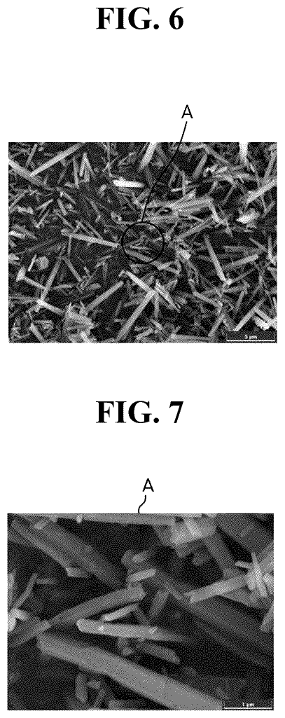

FIG. 6 is a photograph image of an MOF photographed by a scanning microscope, according to an embodiment of the present invention, and FIG. 7 is a photograph image of an enlarged portion A of FIG. 6.

Referring to FIGS. 6 and 7, an MOF according to an embodiment of the present invention may have a particle size of 500 nm to 20 μm, may be formed in a hexagonal pillar shape, and may have an empty space having a diameter of 3.2 nm. Based on such an empty space, various gas molecules may freely move. Also, the empty space may increase a frequency number of reaction with a target organic compound and may provide a high adsorptive force.

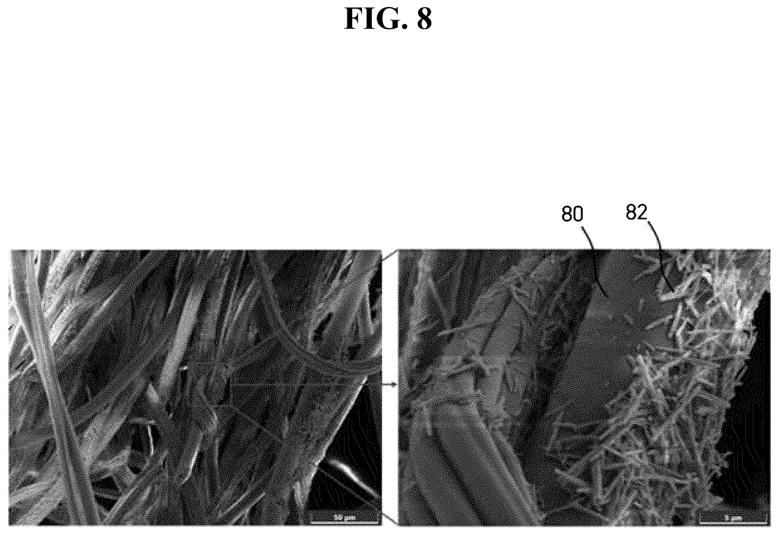

FIG. 8 is a photograph image of a metal-organic framework adsorbed onto a viscose fiber and photographed by a scanning microscope, according to an embodiment of the present invention. In FIG. 8, a reference numeral 80 may refer to a viscose fiber, and a reference numeral 82 may refer to an MOF adsorbed onto the viscose fiber.

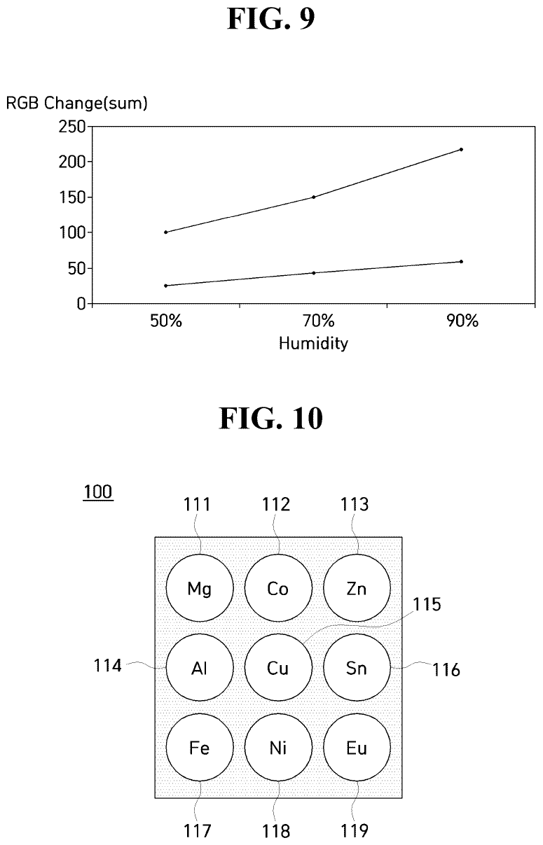

FIG. 9 is a graph showing an RGB color change reactivity with respect to humidity in a general colorimetric sensor manufactured with a color-responsive dye and a colorimetric sensor including an MOF synthesized from metalloporphyrin according to an embodiment of the present invention.

Referring to FIG. 9, G1 is a graph showing an RGB color change reactivity with respect to humidity in the general colorimetric sensor manufactured with a color-responsive dye, and G2 is a graph showing a graph showing an RGB color change reactivity with respect to humidity in the colorimetric sensor including an MOF synthesized from metalloporphyrin according to an embodiment of the present invention

As a result confirmed through an experiment, it may be seen that a colorimetric sensor including an MOF synthesized from metalloporphyrin has a low color change reactivity with respect to humidity as in G2.

FIG. 10 is a diagram for describing a colorimetric sensor array according to an embodiment of the present invention.

Referring to FIG. 10, a colorimetric sensor manufactured by the manufacturing method described above may be manufactured in an array form. For example, in the colorimetric sensor array, a plurality of colorimetric sensors 111 to 119 may be arranged in a matrix form in a tetragonal sheet 110.

In the colorimetric sensors 111 to 119, different metal compounds may be used in a process of manufacturing metalloporphyrin. Therefore, in each colorimetric sensor, metal ions bonded to porphyrin may differ. Accordingly, colorimetric sensors may provide different color change reactivities, and gases corresponding to various volatile organic compounds may be accurately sensed through a difference between color change reactivities.

It will be apparent to those skilled in the art that various modifications and variations can be made in the present invention without departing from the spirit or scope of the inventions. Thus, it is intended that the present invention covers the modifications and variations of this invention provided they come within the scope of the appended claims and their equivalents.

What is claimed is:

1. A method of manufacturing a colorimetric sensor, the method comprising:
a step of obtaining metalloporphyrin;
a step of synthesizing the metalloporphyrin into a metal-organic framework;
a step of adsorbing the metal-organic framework onto a dye catcher sheet; and
a step of drying the dye catcher sheet.

2. The method of claim 1, wherein the metalloporphyrin is a compound where porphyrin is bonded to a metal ion, and
the metal ion is one of a Sn(II) ion, a Cu(II) ion, an Fe(II) ion, a Zn(II) ion, a Ni(II) ion, a Mg(II) ion, a Co(II) ion, an Al(III) ion, an Eu(II) ion, and a Pd(II) ion.

3. The method of claim 2, wherein the porphyrin has a structure where four pyridines are connected to one another in a ring shape, and
each of the four pyridines is a compound including nitrogen (N).

4. The method of claim 1, wherein the step of obtaining the metalloporphyrin comprises:
a step of preparing a porphyrin as a precursor material; and
a step of mixing the porphyrin with a metal compound to obtain the metalloporphyrin, and
the metal compound comprises one metal of tin (Sn), copper (Cu), iron (Fe), zinc (Zn), nickel (Ni), magnesium (Mg), cobalt (Co), aluminum (Al), europium (Eu), and palladium (Pd).

5. The method of claim 1, wherein the step of synthesizing the metalloporphyrin into the metal-organic framework comprises:
a step of generating a compound where the metalloporphyrin is mixed with a linker material;
a step of adding a catalyst to the compound; and
a step of heating the compound with the catalyst added thereto to generate the metal-organic framework.

6. The method of claim 5, wherein the linker material is zirconium chloride (ZrCl₄).

7. The method of claim 5, wherein the catalyst is a benzoic acid.

8. The method of claim 5, wherein the step of generating the compound where the metalloporphyrin is mixed with the linker material comprises a step of adding zirconium chloride (ZrCl₄), which is the linker material, and a solvent to a vial with the metalloporphyrin put therein and uniformly mixing the zirconium chloride (ZrCl₄), the solvent, and the metalloporphyrin with one another through sonication.

9. The method of claim 8, wherein the solvent is one of dimethylformamide, methanol, ethanol, acetone, and isopropanol.

10. The method of claim 5, wherein the step of adding the catalyst to the compound comprises a step of adding a benzoic acid, which is the catalyst, to a vial where a compound of the metalloporphyrin and zirconium chloride (ZrCl₄) which is the linker material is put, and then, mixing the benzoic acid and the compound with each other through sonication.

11. The method of claim 5, wherein the step of heating the compound with the catalyst added thereto to generate the metal-organic framework comprises:
a step of sealing, by using a tape, a vial where a compound of the metalloporphyrin, zirconium chloride (ZrCl₄) which is the linker material, and the benzoic acid which is the catalyst is put, and then, performing a reaction therebetween in an oven; and
a step of drying a product, obtained through a vacuum filtration apparatus after the vial is cooled up to a room temperature, in an oven to generate the dried product as the metal-organic framework.

12. The method of claim 1, wherein the step of adsorbing the metal-organic framework onto the dye catcher sheet comprises:
a step of putting the metal-organic framework into a vial and adding a solvent to the vial to disperse the metal-organic framework in the solvent; and
a step of putting the dye catcher sheet into the vial where the solvent with the metal-organic framework dispersed therein is put, and then, adsorbing the metal-organic framework onto the dye catcher sheet through magnetic stirring.

13. The method of claim 12, wherein the solvent is one selected from among methanol, dimethylformamide (DMF) acetone, isopropanol, and ethanol.

14. The method of claim 12, wherein the dye catcher sheet is a viscose fiber.

15. The method of claim 12, wherein the metal-organic framework is dispersed in the solvent at a weight ratio of 0.1 wt % to 400 wt % of a weight of the dye catcher sheet.
